(19) United States
(12) Patent Application Publication
(10) Pub. No.: US 2024/0165593 A1
(43) Pub. Date: May 23, 2024

(54) CATALYSTS FOR DRY REFORMING OF METHANE AND RELATED METHODS

(71) Applicant: InnoTech Alberta Inc., Edmonton (CA)

(72) Inventors: Partha SARKAR, Edmonton (CA); Aref NAJAFI, Calgary (CA); Farbod SHARIF, Calgary (CA); Volodymyr CHERNYAK, Calgary (CA)

(73) Assignee: InnoTech Alberta Inc., Edmonton (CA)

(21) Appl. No.: 18/512,118

(22) Filed: Nov. 17, 2023

Related U.S. Application Data
(60) Provisional application No. 63/384,337, filed on Nov. 18, 2022.

(51) Int. Cl. B01J 23/755; B01J 23/02; B01J 35/00; B01J 37/02; B01J 37/08; C07C 1/12

(52) U.S. Cl. CPC B01J 23/755; B01J 23/02; B01J 35/0006; B01J 37/0213; B01J 37/08; C07C 1/12

(57) ABSTRACT

Catalysts for dry reforming of methane are provided. In some embodiments, the catalyst comprises a catalyst composition comprising a nickel-copper mixture and a promoter, the promoter comprising barium. The catalyst may be more resistant to sintering and/or coking than conventional catalysts, allowing for use at high temperatures over long periods of time. Related methods for making the catalyst and methods for dry reforming utilizing the catalyst are also provided.

### RELATED APPLICATION

The present application claims priority to U.S. Provisional Patent Application No. 63/384,337 filed Nov. 18, 2022, the entire contents of which are incorporated herein by reference.

### TECHNICAL FIELD

The present disclosure relates to dry reforming of methane. More particularly, the present disclosure relates to catalysts for dry reforming of methane and related methods.

### BACKGROUND

Greenhouse gases (GHG) allow shorter wavelength solar radiation to pass to the earth's surface but then absorb and re-radiate the longer wavelength (infrared) radiation generated by the hot earth's surface, causing an increase in atmospheric temperature known as the “greenhouse effect”. Carbon dioxide (CO₂) is the most prominent greenhouse gas arising from anthropogenic activities. In addition, methane (CH₄) is another GHG that is even more potent than CO₂ in increasing the atmospheric temperature. The mitigation and utilization of greenhouse gases is increasingly critical to combat global warming and climate change.

Dry reforming of methane (DRM) is a process that converts the two primary GHGs (CO₂ and CH₄) into synthesis gas (H₂ and CO) via Equation (1) below:

CH₄+CO₂→2CO+2H₂, ΔHº=247.3 k mol⁻¹,

ΔGº=61 770-67.32 T kJ mol⁻¹

The synthesis gas produced by DRM has a H₂/CO ratio of about 1.0 due to the simultaneous occurrence of the reverse water gas reaction (RWGR), as shown by Equation (2):

CO₂+H₂→CO+H₂O, ΔHº=−41.2 kJ mol⁻¹,

ΔGº=−8545+7.48 T kJ mol⁻¹

Thus, DRM not only utilizes CO₂ and CH₄ to reduce the emission of GHG but also produces an industrially important synthesis gas (“syngas”), which can be used to manufacture value-added products. Syngas with a H₂/CO ratio of 1.0 can be used for the synthesis of long-chain hydrocarbons or oxygenate chemicals such as acetic acid, dimethyl ether and oxo-alcohols. In addition, the H₂/CO ratio can be adjusted to 2.0 via an additional water-gas shift step to allow the syngas to be used for methanol and Fischer-Tropsch synthesis.

Currently, DRM is not regarded as a mature industrial process, despite its potential economic and environmental benefits. One of the major challenges for industrial implementation of DRM is the selection of a suitable catalyst to convert CO₂ and CH₄ to H₂ and CO via Equation (1). DRM was initially developed using nickel (Ni) and cobalt (Co) catalysts and has been tested with a variety of noble and non-noble metals. However, since DRM is a high-temperature process, non-noble metal catalysts typically suffer from deposition of carbon on the catalyst surface (referred to as coke formation or “coking”) as well as densification/sintering during operation, leading to a reduction of the active catalyst surface area and deterioration of catalyst performance. Replacement of the catalyst can be costly. Some noble metal catalysts (iridium, rhodium, ruthenium, platinum and palladium) have been found to be more resistant to coke formation (Pakhare et al. A review of dry (CO₂) reforming of methane over noble metal catalysts. Chem. Soc. Rev. 43 (2014) 7813-7837) but such metals have high costs and low availability. The addition of a co-feed of large amounts of steam may eliminate severe carbon formations (Mortensen et al. Industrial scale experience on steam reforming of CO2-rich gas. Appl. Catal. A 495 (2015) 141-151); however, steam generation is both capital and energy intensive.

### SUMMARY

In one aspect, there is provided a catalyst for dry reforming of methane, comprising: a catalyst composition comprising a nickel-copper mixture and a promoter, the promoter comprising barium.

In some embodiments, nickel is present in the nickel-copper mixture at an atomic concentration of between about 20% and about 85%.

In some embodiments, the barium is present in the catalyst composition at an atomic concentration of between about 0.1 and about 20%.

In some embodiments, the barium is homogenously distributed in the nickel-copper mixture.

In some embodiments, the nickel-copper mixture further comprises cobalt.

In some embodiments, the catalyst composition further comprises at least one oxide additive.

In some embodiments, the at least one oxide additive comprises zirconium oxide (ZrO₂), cerium oxide (CeO₂), praseodymium oxide (Pr6O11), a solid solution thereof, or a combination thereof.

In some embodiments, the at least one oxide additive is present in the catalyst composition at a total volume percentage of about 5% to about 60%.

In some embodiments, the at least one oxide additive is doped with aliovalent cations.

In some embodiments, the promoter further comprises magnesium.

In some embodiments, the catalyst further comprises a support for the catalyst composition.

In some embodiments, the support comprises activated alumina or a soft fibrous insulation.

In some embodiments, the catalyst composition is substantially homogenously distributed on the surface of the support.

In another aspect, there is provided a method for making a catalyst, comprising: providing solutions of nickel, copper, and barium; providing a support; impregnating the support with the solutions to form an impregnated support; heat treating the impregnated support to form a heat-treated support; and activating the heat-treated support to form the catalyst.

In some embodiments, the step of impregnating the support with the solutions utilizes a vacuum or incipient wetness impregnation technique.

In some embodiments, activating the heat-treated support comprises reducing the heat-treated support in the presence of hydrogen.

In some embodiments, the method further comprises providing at least one oxide additive solution and wherein the step of impregnating the support with the solutions further comprises impregnating the support with the at least one oxide additive solution.

In some embodiments, the method further comprises providing a cobalt solution and wherein the step of impregnating the support with the solutions further comprises impregnating the support with the cobalt solution.

In another aspect, there is provided a method for dry reforming of methane, comprising: providing a catalyst comprising: a catalyst composition comprising a nickel-copper mixture and a promoter, the promoter comprising barium; and contacting a feed gas with the catalyst, the feed gas comprising methane and carbon dioxide.

In some embodiments, the step of contacting the feed gas with the catalyst is performed for at least 300 hours.

Other aspects and features of the present disclosure will become apparent, to those ordinarily skilled in the art, upon review of the following description of specific embodiments of the disclosure.

### BRIEF DESCRIPTION OF THE DRAWINGS

Some aspects of the disclosure will now be described in greater detail with reference to the accompanying drawings. In the drawings:

FIG. 1 is a flowchart of an example method for making a catalyst, according to some embodiments;

FIG. 2 is a flowchart of an example method for dry reforming of methane, according to some embodiments;

FIG. 3 is a series of photographs showing: (a) a soft fibrous insulation support impregnated with a catalyst composition and air dried (shown placed inside a quartz reactor tube); (b) the dried impregnated catalyst after heat treatment and hydrogen reduction; and (c) the heat-treated catalyst after a dry reforming of methane experimental run at 700° C.;

FIG. 4 is a series of photographs showing: (a) pure activated alumina; (b) the activated alumina following catalyst impregnation and drying; (c) the impregnated and dried catalyst following heat treatment; and (d) the heat treated catalyst after hydrogen reduction;

FIG. 5 is a schematic block diagram of an experimental system used to perform dry reforming of methane experiments;

FIG. 6 is a graph showing gas compositions at the reactor outlet of the experimental system of FIG. 5 as a function of operation temperature, using the catalyst of FIG. 3(b);

FIG. 7 is a graph showing gas compositions at the reactor outlet of the experimental system of FIG. 5 as a function of flow rate, using the catalyst of FIG. 3(b);

FIG. 8 is a graph showing gas compositions at the reactor outlet of the experimental system of FIG. 5 as a function of time, using the catalyst of FIG. 3(b);

FIG. 9 is a graph showing gas compositions at the reactor outlet of the experimental system of FIG. 5 as a function of time, using the catalyst of FIG. 4(d); and

FIGS. 10A-10F are a series of electron micrographs showing an experimental catalyst on an activated alumina support following dry methane reforming runs, at magnifications of 25X, 75X, 500X, 1300X, 10000X, and 30000X, respectively.

### DETAILED DESCRIPTION

Generally, the present disclosure provides a catalyst for dry reforming of methane. Related methods for making the catalyst and methods for dry reforming utilizing the catalyst are also provided.

As used herein the terms “a”, “an”, and “the” may include plural referents unless the context clearly dictates otherwise.

As used herein, “dry reforming of methane” (DRM) and “carbon dioxide reforming” are used interchangeably herein to refer to the conversion of carbon dioxide and methane into hydrogen and carbon monoxide via Equation (1) above. However, it will be understood that the CO₂ may be reformed in the presence of hydrocarbons other than methane and “dry reforming of methane” is intended to be inclusive of such other reactions. In addition, the catalysts described herein may be useful for other applications and their use is not limited to only dry reforming of methane.

The catalyst comprises a catalyst composition. The catalyst composition may comprise a nickel-copper (Ni—Cu) mixture and a catalyst promoter. The nickel-copper mixture may also be referred to as a nickel-copper catalyst. In some embodiments, the nickel-copper mixture is in the form of a solid solution. The solid solution may be a complete solid solution such that the Ni and Cu are completely soluble with one another in the solid state. In some embodiments, nickel is present in the nickel-copper mixture at an atomic concentration of at least about 20%, at least about 40%, or at least about 50%. In some embodiments, nickel is present in the nickel-copper mixture at an atomic concentration of about 85% or less, about 60% or less, or about 50% or less. In some embodiments, nickel is present in the nickel-copper mixture at an atomic concentration of between about 20% and about 85%, or between about 40% and about 60%, or approximately 50%. The copper may be present in the nickel-copper mixture at an atomic concentration of at least about 15%, at least about 40%, or at least about 50%. In some embodiments, the copper is present in the nickel-copper mixture at an atomic concentration of about 80% or less, about 60% or less, or about 50% or less. In some embodiments, the copper is present in the nickel-copper mixture at an atomic concentration of between about 15% to about 80%, or between about 40% and about 60%, or approximately 50%.

The catalyst promoter may comprise barium (Ba). In some embodiments, the promoter is present in the catalyst composition at an atomic concentration of at least about 0.1%, at least about 1%, or at least about 2%. In some embodiments, the promoter is present in the catalyst composition at an atomic concentration of about 20% or less, about 5% or less, about 3% or less, or about 2% or less. In some embodiments, the promoter is present in the catalyst composition at an atomic concentration of between about 0.1 and about 20%, or between about 1% and about 5%, between about 1% and 3%, or approximately 2%. In some embodiments, the promoter consists solely of Ba. In other embodiments, the promoter comprises a mixture of Ba and magnesium (Mg). The promoter may be homogenously distributed in the Ni—Cu mixture. The term “homogenously distributed” in this context refers to substantially even distribution of the promoter throughout the Ni—Cu mixture (proportional to the atomic concentrations as discussed above), although it will be understood that distribution may not be perfectly uniform and minor variations are possible.

In some embodiments, the catalyst further comprises a support for the catalyst composition. The support may comprise a solid material having a high surface area. In some embodiments, the support comprises a porous material including, but not limited to, activated alumina. In other embodiments, the support comprises a non-porous material. In some embodiments, the non-porous material is a soft fibrous insulation, such as a ceramic insulation. A non-limiting example of a ceramic insulation material is Al₂O₃—SiO₂ (alumina/silica fiber). Alternative support materials include α-Al₂O₃ (α-aluminum oxide), ZrO₂ (zirconia), stabilized-ZrO₂, CeO₂ (cerium oxide/ceria), doped-CeO₂, and Spinel (MgAl₂O₄). Other suitable materials may include materials with high thermal conductivity such as aluminum nitride (AlN) and boron nitride (BN), or electrically conducting ceramics such as silicon carbide (SiC). In other embodiments, the support may comprise any other suitable material.

The catalyst composition may be impregnated on the surface of the support and, in the case of porous supports, within the pores on the pore surfaces thereof. The term “impregnate” in this context refers to adsorption, adhesion, bonding, or any other physical or chemical association between the Ni—Cu—Ba composition and the surface of the support. The combination of the catalyst composition and the support forms a nanoscale catalyst with a high surface area.

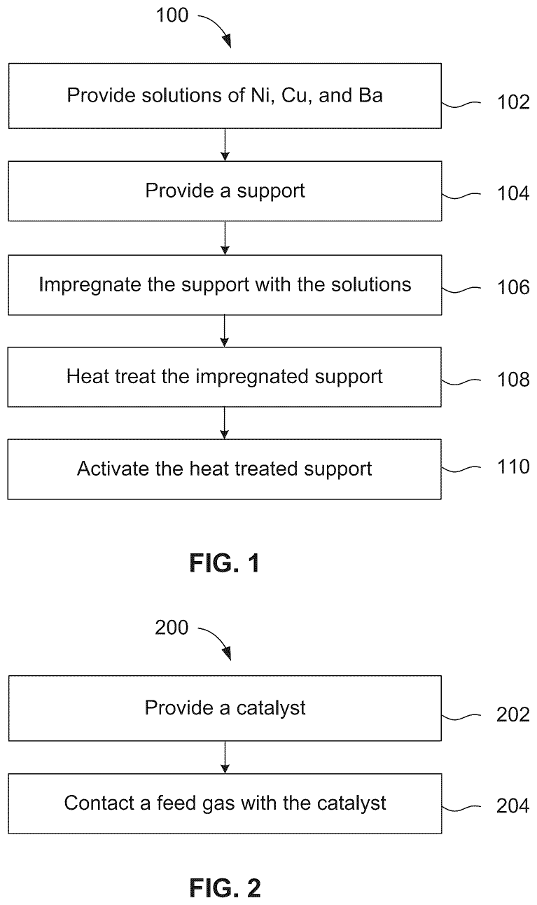

The distribution of the catalyst composition on the surface of the support may be homogenous. The term “homogenous” in this context refers to an approximately even distribution of Ni, Cu, and Ba (proportional to their individual atomic concentrations as discussed above) across the available surface area of a given support, although it will be understood that the distribution may not be perfectly uniform. The homogenous distribution of the catalyst composition may be achieved using a suitable impregnation method, such as a method utilizing a vacuum and/or incipient wetness impregnation technique. An example method is shown in FIG. 1 and discussed below.

In other embodiments, the catalyst may be a support-free catalyst. In some embodiments, the support-free catalyst may be in the form of a powder or an agglomerate of powder (e.g. a granular form), in the absence of a support. For example, the powder or granules may comprise salts of Ni, Cu, and Ba (e.g. Nickel Nitrate, Copper Nitrate, Barium Nitrate), oxides of Ni, Cu, and Ba (e.g. NiO, CuO, BaO) or a mixture of salts and oxides (e.g. NiO with Copper Nitrate and Barium Nitrate). In these embodiments, the Ba promoter may be homogenously distributed throughout the Ni—Cu mixture in each powder or granule particle. In some embodiments, the powder or granules may be porous in nature. Porous powder or granules have higher surface areas, thereby increasing catalytic activity.

In some embodiments, the support-free catalyst may further comprise a second phase material. The second phase material may be inert to the catalytic cations and may comprise, for example, zirconia, ceria, boron nitrate (BN), or aluminum nitrate (AIN). Optionally, the second phase material may further comprise boron carbide (BC). The concentration of the second phase may vary and may be in the range of about 1% to about 45% by volume in some embodiments. The inclusion of the second phase may help to retard the densification (sintering) of the porous powder or granules.

In yet other embodiments, the catalyst composition (e.g. Ni/Cu/Ba), or the catalyst composition plus a second phase material, may be coated on an interior surface of a reactor. In these embodiments, the surface of the reactor itself may act as a support for the catalyst composition. In some embodiments, the reactor is a microchannel reactor. An example of a microchannel reactor is described in Tonkovich et al. “Microchannel Process Technology for Compact Methane Steam Reforming”, Chem. Eng. Sci 59 (22-23) p. 4819-4824 2004, incorporated herein by reference.

As demonstrated in the Examples below, the catalysts disclosed herein display strong resistance to coke formation and sintering during DRM operations. Ni has a high catalytic activity, whereas the catalytic activity of Cu is low or nearly zero. A pure Ni catalyst results in significant coke formation during a DRM process. The addition of Cu to the catalyst composition dilutes the catalytic activity of pure Ni, thereby reducing coke formation. Without being limited by theory, it is believed the Ba promoter introduces basic sites throughout the Ni—Cu catalyst mixture, which further reduces coking and improves catalyst performance. The primary source of coke formation in DRM at high temperatures is methane decomposition, which converts methane to hydrogen and elemental carbon. Methane decomposition is known to occur on acid sites and, thus, increasing the basicity of the catalyst is thought to provide resistance against carbon formation. In addition, it is believed that the basic sites introduced by the Ba promoter favor adsorption of the mildly acidic CO₂ (i.e., Lewis acidity) to the catalyst surface adjacent to Ni—Cu sites. Adsorption of CO₂ adjacent to Ni—Cu sites increases formation of carbonate species, which supplies surface oxygen to oxidize the surface carbon to carbon monoxide (CO), further reducing coke formation. The substantially homogenous distribution of the Ba promoter in the Ni—Cu catalyst therefore helps to ensure that the basic sites are evenly distributed across the surface of the catalyst composition to inhibit overall coke formation. It is also hypothesized that the Ba promotor reduces sintering of the catalyst, which is supported by the experiments in the Examples below.

In some embodiments, the Ni—Cu mixture of the catalyst composition further comprises cobalt (Co). In terms of catalytic activity, Co falls between Ni (high activity) and Cu (low to no activity). Co can be used to replace a portion of the Ni in the Ni—Cu mixture to reduce the catalytic activity of the composition or it can be used to replace a portion of the Cu to increase catalytic activity. Thus, by adjusting the proportions of Ni, Cu, and Co, the composition can be fine-tuned to provide a balance between reaction efficiency and reduction/elimination of coking. The amount of the Ba promoter in the Ni—Cu—Co catalyst composition can also be adjusted as another parameter.

In some embodiments, the catalyst composition further comprises at least one oxide additive. Non-limiting examples of oxide additives include zirconium oxide/zirconia (ZrO₂), cerium oxide/ceria (CeO₂), praseodymium oxide/praseodymia (Pr6O11), a solid solution thereof, and/or a combination thereof. In some embodiments, the oxide additive (e.g. ZrO₂, CeO₂, Pr6O11) is doped with aliovalent cations. Non-limiting examples of aliovalent cations include trivalent cations such as Y³⁺ (yttrium), Gd³⁺ (gadolinium) and Sc³⁺ (scandium), and divalent cations such as Ca²⁺ (calcium) and Mg² (magnesium).

The oxide additive may be added to the catalyst composition at a volume percentage of about 5% to about 95% (i.e. about 5-95 mL of additive to every 100 mL of Ni—Cu—Ba or Ni—Cu—Co—Ba). The oxide additive may therefore be present in the final catalyst composition at a total volume percentage of about 5% to about 60%. The oxide additive(s) may be present in the catalyst composition as a separate phase or phases from the Ni—Cu or Ni—Cu—Co mixture.

The oxide additive(s) may provide additional basicity to the catalyst composition, which reduces coking as discussed above. The oxide additive phase(s) may also function as oxygen ion-conductors to provide oxygen storage capacity, which may contribute to the activation of CO₂. Mobile oxygen may oxidize carbon to CO to prevent carbon formation on the surface of the catalyst. In addition, the presence of the oxide additives as a separate phase in the Ni—Cu or Ni—Cu—Co mixture may prevent or retard grain growth and densification of the catalyst composition. Therefore, the addition of oxides may improve catalyst performance by preventing both coking and sintering. Aliovalent cation doping of the oxides may further enhance their oxygen ion conductivity and storage capacity by creating oxygen vacancies.

In other embodiments, the catalyst may comprise any other suitable materials and/or additives and embodiments are not limited to only the components disclosed herein.

FIG. 1 is a flowchart of an example method 100 for making a catalyst, according to some embodiments.

At block 102, solutions of nickel, copper, and barium are provided. As used herein “providing” refers to making, acquiring, purchasing, or otherwise obtaining a material or substance. In some embodiments, the nickel, copper, and barium are provided as nitrate solutions such as, for example, Nickel Nitrate Hexahydrate, Cupric Nitrate Hemi(pentahydrate), and Barium Nitrate, respectively. The solutions may be aqueous solutions, or a solution in any other suitable solvent, and may be at any suitable concentration. Optionally, a mixture of Barium Nitrate and Magnesium Nitrate may be provided instead of Barium Nitrate.

The Ni, Cu, and Ba solutions may be combined to form a solution mixture prior to contacting the support at block 106 below. Specified quantities of the solutions may be combined to achieve the final atomic concentrations of Ni, Cu, and Ba described above. Water (or another solvent) may be added to the solution mixture to bring the Ni, Cu, and Ba to desired concentrations. In some embodiments, the Ni and Cu solutions are combined first, followed by addition of the Ba solution. In other embodiments, the solutions are combined in any other suitable order.

In some embodiments, a solution of cobalt is also provided and combined with the Ni, Cu, and Ba solutions. In some embodiments, the Ni, Cu, and Co solutions may be combined first, followed by addition of Ba. The Co may be provided as a nitrate solution or any other suitable solution.

In some embodiments, at least one oxide additive solution is also provided. It will be understood that the “oxide additive solution” in this context may be a solution containing the desired cation (e.g. Zr+ or Ce+) in the form of a salt or any other suitable form, and the actual oxide may be formed via the heat treatment step of block 108 discussed below. The oxide additive solution may be combined with the Ni, Cu, Co (optionally) and Ba solutions or may be used in a sequential impregnation technique as discussed below. The oxide additive may be any of the additives discussed above. In some embodiments, the oxide additive solution is added to the Ni and Cu mixture (and Co if used), before or after addition of Ba.

At block 104, a support is provided. The support may comprise any of the support materials described above including, but not limited to, activated alumina or soft fibrous insulation.

At block 106, the support is impregnated with the solutions to form an impregnated support. The impregnation step may utilize a vacuum and/or incipient wetness technique. In other embodiments, the impregnation step may utilize any other suitable technique. As discussed above, the support may be impregnated with a solution mixture of Ni, Cu, and Ba (and optionally Co and/or one or more oxide additives) such that the support is impregnated with all of the catalyst components substantially simultaneously.

In other embodiments, the impregnation step may comprise sequentially impregnating the support with each component individually or solution mixtures of two or more components. In a sequential technique using vacuum impregnation, the individual solution concentration of each component, and the absorbed amount, may be calibrated to maintain the desired Ni—Cu—Ba ratio. In addition, an additional heat treatment (or other steps) may be performed between each impregnation step to reduce the risk of the already absorbed components dissolving into the subsequent component solution.

In embodiments in which the catalyst includes at least one oxide additive (or at least one doped oxide additive), the support may be impregnated all at once with a solution containing all of the cations in the appropriate ratios (e.g. Ni, Cu, Ba and Zr, Ce, doped Zr, or doped Ce). An advantage to this technique is that it provides atomic level mixing and, thus, all components may be homogenously mixed. The impregnated support may then be subjected to heat treatment and reduction/activation at blocks 108 and 110 below. Oxides (e.g. ZrO₂ or CeO₂) will form during the heat treatment and, during reduction/activation, Ni and Cu will reduce to metal while the oxides will not reduce. The oxides may then retard the densification of catalyst particles in operation at high temperatures as discussed above.

Alternatively, the support may be impregnated sequentially with the Ni/Cu/Ba and the additive. One option for sequential impregnation would be to first impregnate the support with a solution of the additive (e.g. a Zr-salt with a dopant or a Ce-salt with a dopant), followed by heat treatment, and then impregnating the support with a solution of Ni/Cu/Ba. In this scenario, the oxide additive (e.g. ZrO₂ or CeO₂) may enhance the surface area of the support, in addition to its role in retarding sintering and densification. A second option for sequential impregnation would be to first impregnate the support with a solution of the additive (e.g. Zr or Ce), followed by heat treatment, and then impregnating the support with a solution of Ni/Cu/Ba plus an additional portion of the additive. In this scenario, the additive is mixed with the Ni/Cu/Ba solution after impregnation of the oxide (e.g. ZrO₂ or CeO₂), which may provide better protection from densification.

The impregnated support may then be removed from the impregnation apparatus and dried. The support may be dried in air or in the presence of a suitable inert gas. The support may be dried at room temperature or may be heated to a higher temperature. In some embodiments, the impregnated support is dried in two or more stages at increasing temperatures. For example, the support may be dried at room temperature for a first time period, then dried at 80° C. for a second time period, and then dried at 120° C. for a third time period. The first, second, and third time periods may be any suitable amount of time e.g. between about 1 to 2 hours each. In other embodiments, the support may be dried by any other suitable means.

At block 108, the impregnated support is heat treated. The heat treatment may be at a temperature between about 500° C. and about 850° C., between about 550° C. and about 650° C., or at approximately 600° C. The support may be heat treated in air, or in the presence of a suitable inert gas, for a suitable period of time. In some embodiments, the heat treatment is between about 3 hours and about 12 hours. As one specific example, the impregnated support may be heated at 600° C. for about 5 hours in air. In some embodiments, the heat treatment temperature may be approximately the same (or slightly higher) than the intended temperature for the dry reforming operation in which the catalyst will be used. Temperatures for dry reforming reactions are discussed in more detail below.

At block 110, the heat-treated support is activated. Activation may comprise reduction of the heat-treated support. In some embodiments, reducing the support comprises heat treating the support in the presence of hydrogen gas (H₂). The temperature may be between about 500° C. and about 700° C., between about 550° C. and about 650° C., or at approximately 600° C. The H₂ concentration may be between about 1% and about 10%, between about 3% and about 7%, or approximately 5%. The activation/reduction step may be between about 1 and about 10 hours, between about 3 and about 7 hours, or approximately 5 hours. As one specific example, the impregnated support may be activated at 600° C. in 5% hydrogen for about 5 hours.

Following the steps at block 110, the catalyst is now ready for use. As discussed above, production of the catalyst by solution impregnation techniques allows for the substantially homogenous distribution of the catalyst composition on the support. However, it will be understood that it may be possible for other techniques to produce a homogenous catalyst and embodiments are not limited to only the techniques disclosed herein.

As discussed above, support-free catalysts are also contemplated including catalysts in the form of powder or granules. In these embodiments, the catalysts may be prepared by mixing salts and/or oxides of the Ni, Cu, Ba (and other optional components) together. In other embodiments, the catalyst may be prepared by coating a mixture of Ni/Cu/Ba (and other optional components) on an interior surface of a reactor, using a solution coating method or any other suitable technique.

FIG. 2 is a flowchart of an example method 200 for dry reforming of methane.

At block 202, a catalyst is provided. The catalyst may be any embodiment of the catalyst discussed above. In some embodiments, providing the catalyst comprises making the catalyst via the method 100 of FIG. 1. In other embodiments, the catalyst may be provided by any other suitable means.

At block 204, a feed gas is contacted with the catalyst. The feed gas may comprise carbon dioxide and methane. Alternatively, the feed gas may comprise carbon dioxide and any other suitable hydrocarbon. The ratio between the CH₄ and CO₂ may be about 1:1 or any other suitable ratio. It will also be understood that the feed gas may not be purely CH₄ and CO₂ and may contain small amounts of other impurities.

In some embodiments, contacting the feed gas with the catalyst comprises flowing the feed gas through the catalyst. For example, the catalyst may be contained within a suitable reactor and the feed gas may be flowed through the reactor. The flow rate may be constant or variable. In some embodiments, the flow rate is selected based on the particular reactor design and the type of support used in the catalyst. For example, the feed gas may have a flow rate of between about 10 and about 200 SCCM (standard cubic centimeters per minute), or between about 20 and about 100 SCCM. In other embodiments, the flow rate may be any other suitable rate. In some embodiments, a single feed gas stream comprising both CH₄ and CO₂ is introduced into the reactor. In other embodiments, separate streams of CH₄ and CO₂ are introduced into the reactor to form the feed gas therein.

In some embodiments, the feed gas is heated as it contacts the catalyst. For example, the reactor may be heated to heat the gas flowing therethrough. The reactor may be heated to a temperature of at least about 600° C., or between about 600° C. and about 900° C., or between about 600° C. and about 800° C. In other embodiments, the contacting step may be performed at any other suitable temperature. Optionally, the feed gas may be preheated to a suitable temperature before entering the reactor. The preheating temperature may be lower than the reactor temperature and low enough to avoid any chemical reaction or decomposition of the feed gas. In some embodiments, separate streams of CH₄ and CO₂ are preheated and then introduced into the reactor.

As the feed gas contacts the catalyst, at the selected temperature, the dry reforming reaction between methane and carbon dioxide is catalyzed, resulting in the conversion of the methane and carbon dioxide to hydrogen and carbon monoxide via Equation (1) above. The syngas (H₂ and CO) may then be collected and processed and/or used for downstream applications.

As demonstrated in the Examples below, the dry reforming step at block 204 may be performed for at least 300 hours without deterioration of catalyst performance. Thus, embodiments of the catalysts and methods disclosed herein allow dry reforming reactions to be performed at high temperatures for extended periods of time while maintaining stability of the catalyst. The catalyst may therefore not need to be replaced or regenerated as frequently as conventional DRM catalysts, leading to improved efficiency and cost savings.

Without any limitation to the foregoing, the catalysts and methods disclosed above are further described by way of the following examples.

### Example 1—Dry Reforming of Methane Experiments

### 1.1 Catalyst Preparation by Solution Impregnation

A binary catalyst composition comprising a Ni—Cu mixture with Ba-promoter was used for the experiments described herein. Catalysts were produced using both Al₂O₃—SiO₂ soft fibrous insulation and activated alumina as supports. The catalysts were prepared according to the method of FIG. 1.

In the case of the catalyst using the soft insulation support, 0.5 M stock solutions of Nickel Nitrate Hexahydrate, Cupric Nitrate Hemi(pentahydrate) and 0.4 M Barium Nitrate were prepared. 100 ml of a 0.05 M solution mixture with a Ni:Cu ratio 1:1 was prepared using the stock solutions and water, and 0.2 ml of the Barium Nitrate 0.4 M stock solution was added. This solution mixture was placed in a magnetic stirrer to keep it well mixed and was used for impregnation of the soft fibrous insulation using a vacuum and/or incipient wetness impregnation technique.

In the case of the catalyst using the activated alumina support, 100 ml of a 0.1 M solution mixture with a Ni:Cu ratio 1:1 was prepared using the stock solutions and water, and 0.4 ml of the Barium Nitrate 0.4 M stock solution was added. This solution mixture was used to impregnate activated alumina using a vacuum and/or incipient wetness impregnation technique.

The impregnated supports were removed and initially dried in air for 2 hours and then moved to a drier at 80° C. for 2 hours, followed by drying at 120° C. for 1 hour. The now dried catalyst was transferred to a furnace and heat-treated at 600° C. for 5 hours in air. Following the heat treatment, the catalyst was reduced/activated by heat treatment at 600° C. in 5% hydrogen flow for 5 hours.

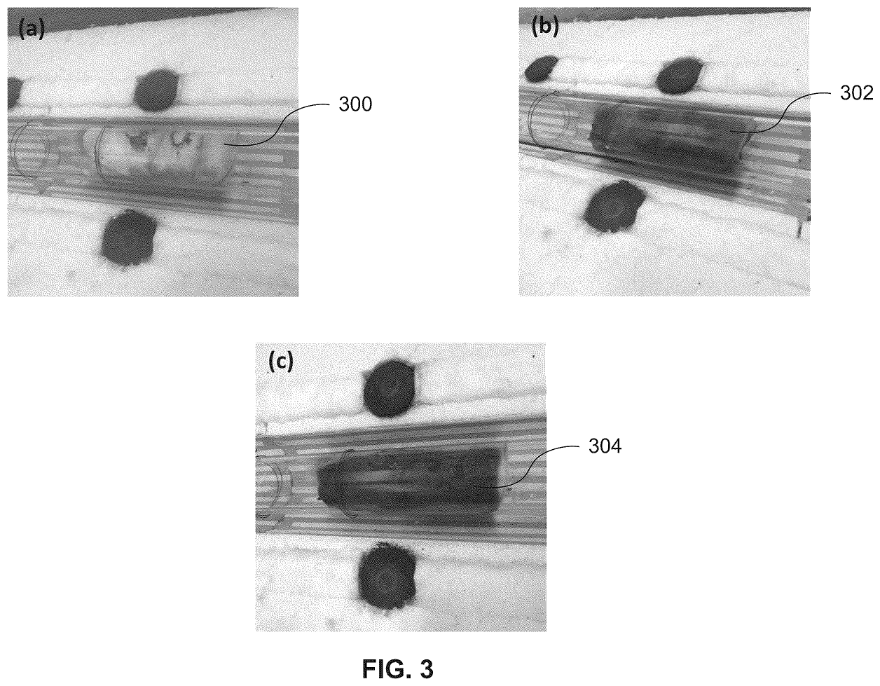

FIG. 3(a) shows an impregnated soft insulation support 300 prior to heat treatment. FIG. 3(b) shows a catalyst 302 formed by heat treating the impregnated support and reduction in H₂. The catalyst 302 in FIG. 3(b) is ready for use. FIG. 3(c) shows the utilized catalyst 304 following a DRM operation at 700° C.

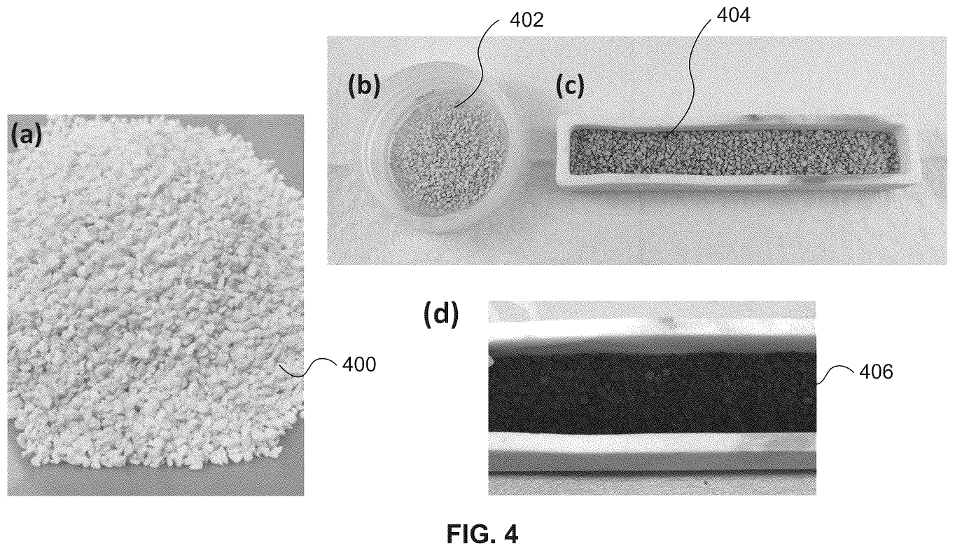

FIG. 4(a) shows untreated activated alumina 400. FIG. 4(b) shows impregnated activated alumina 402 after impregnation and drying and FIG. 4(c) shows heat treated impregnated activated alumina 404. FIG. 4(d) shows the final catalyst 406 after activation/reduction in H₂. The catalyst 406 in FIG. 4(d) is ready for use.

### 1.2 DRM Experimental Setup

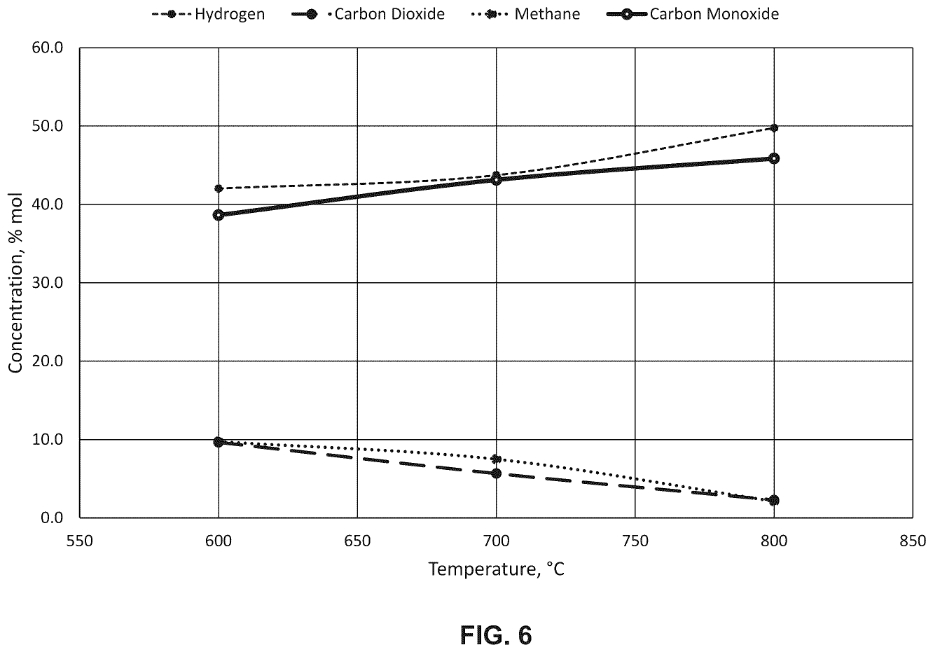

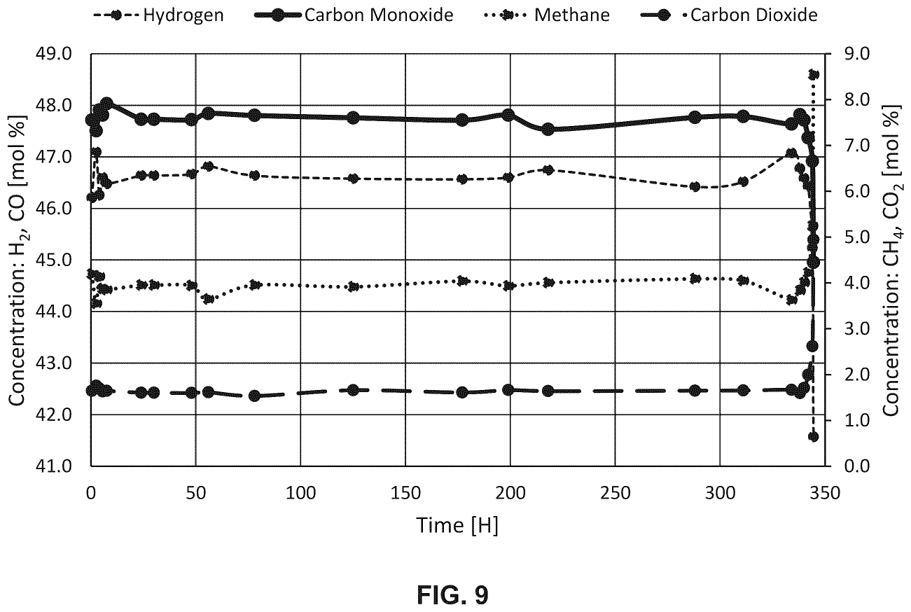

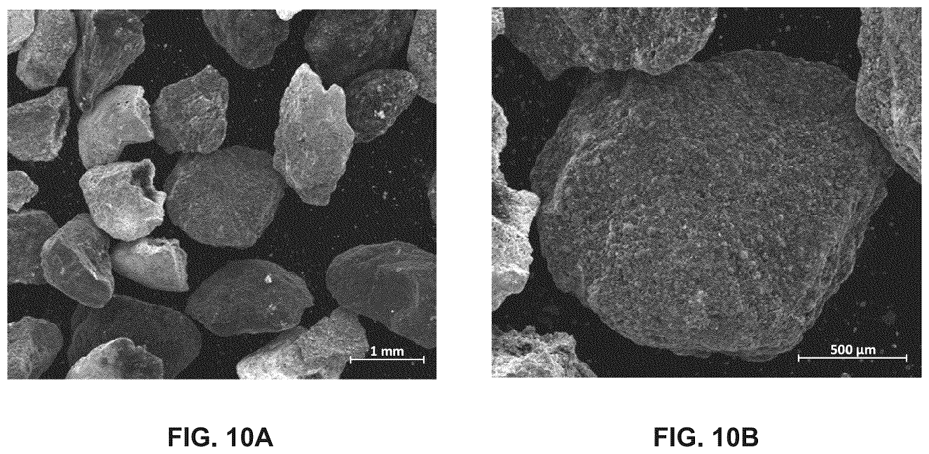

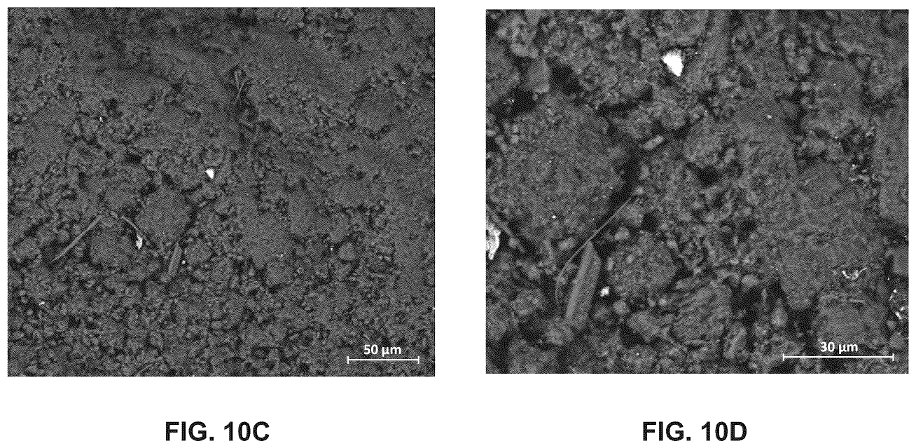

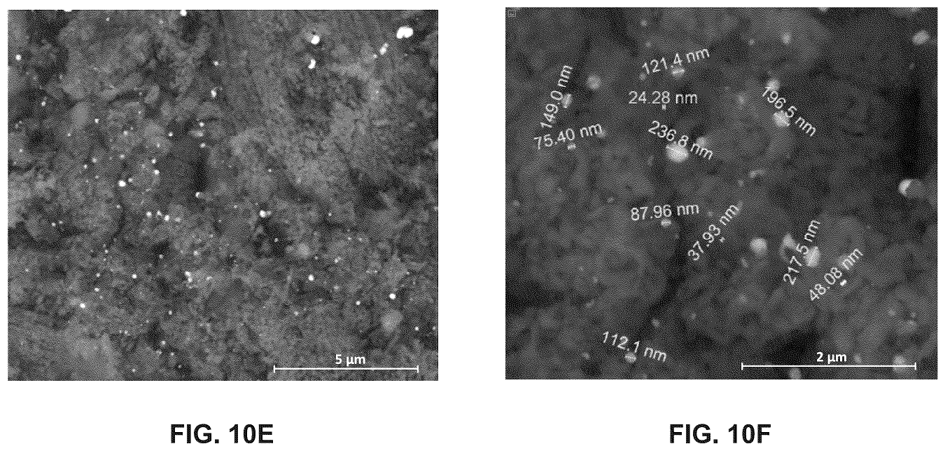

The DRM experiments in FIGS. 6-8 were conducted using the catalyst of FIG. 3(b) supported on the soft fibrous insulation. The experiments in FIGS. 9 and 10 were conducted with the catalyst of FIG. 4(d) supported on activated alumina. The feed gas was a 1:1 mixture of CH₄ and CO₂ for all experiments.

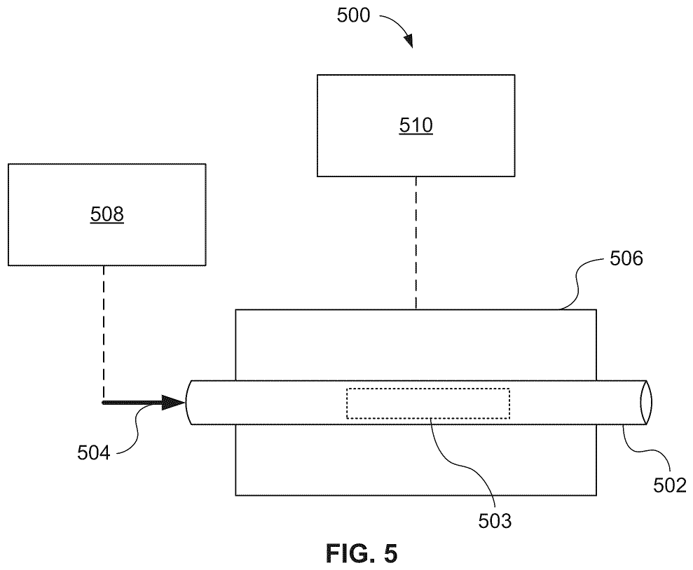

FIG. 5 is a block diagram schematic of an experimental system 500 used in the experiments of FIGS. 6-8. The system 500 included a quartz reactor tube 502 (with catalyst 503 received therein), an injection assembly 504 to introduce the feed gas into the reactor tube 502, and a high temperature furnace 506 to heat the reactor tube 502 as the feed gas flows therethrough. The system 500 also included inlet gas flow controllers 508 to control the flow rate of the feed gas and a temperature controller 510 to control the operating temperature. To evaluate the effects of different parameters on the conversion of CH₄ and CO₂ to H₂ and CO, gas compositions were measured at the outlet of the reactor tube 502 of the system 500.

### 1.3 DRM Experimental Results

FIG. 6 shows the results of DRM runs using a 20 SCCM flow rate of the feed gas and three different temperatures, 600° C., 700° C. and 800° C., to estimate the conversion of CH₄ and CO₂ to H₂ and CO as a function of temperature. Equation (1) shows that DRM is an endothermic process and temperature increases enhance DRM conversion, which is reflected in FIG. 6. The gas compositions of FIG. 6 are also tabulated in Table 1 below:

| | Temperature (° C.) | | |
|---|---|---|---|
| Gas Component | 600 | 700 | 800 |
| CO2 (mol %) | 9.6 | 5.7 | 2.3 |
| CH4 (mol %) | 9.7 | 7.5 | 2.1 |
| CO (mol %) | 38.6 | 43.1 | 45.9 |
| H2 (mol %) | 42.2 | 43.7 | 49.7 |

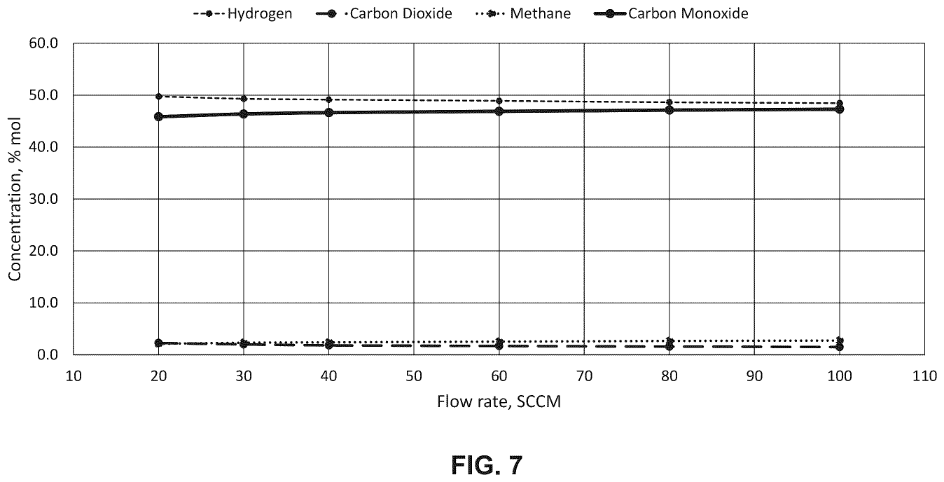

FIG. 7 shows the results of DRM runs at 800° C. and different flow rates varying from 20 to 100 SCCM. The results in FIG. 7 show that, in this flow rate range, conversion is independent of flow rate and is close to 96%.

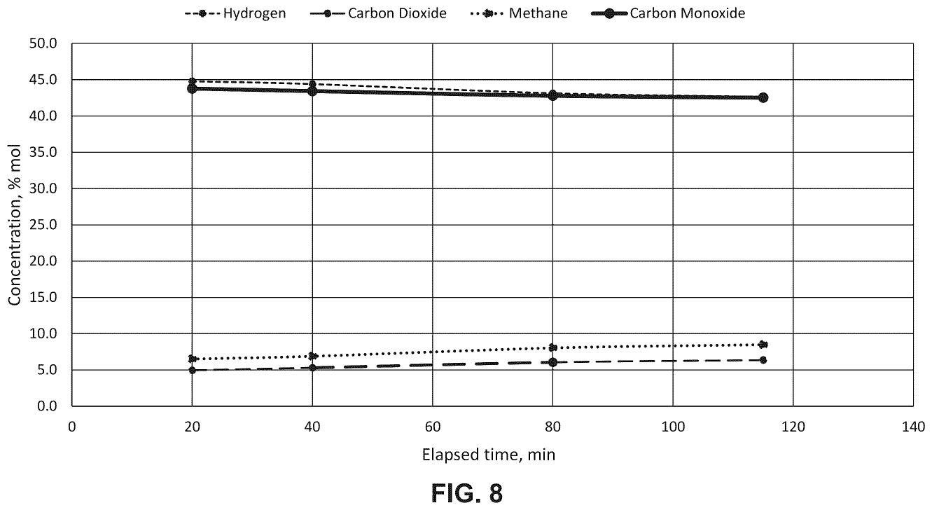

FIG. 8 shows the results of DRM runs at 700° C. over 120 minutes, demonstrating that the catalyst is stable over time. To evaluate long-term stability of the catalyst, a DRM run was conducted for over 300 hours at 800° C. at a flow rate of 20 SCCM for the majority of the run, and a variable flow rate near the end. The results of the 300 hour experiment are shown in FIG. 9 and demonstrate that the catalyst's performance is very stable over an extended period of time. The catalyst's performance does not deteriorate over time and the conversion of CH₄ and CO₂ to H₂ and CO remains over 90% past the 300 hour mark. It can be concluded from the experiments in FIGS. 6-9 that the tested catalysts did not suffer from the coking and sintering problems, which allowed them to maintain stable performance over a long period of time.

FIGS. 10A-10F are electron micrographs showing the catalyst on activated alumina support following DRM runs. The pictures show that there is no indication of sintering between catalyst particles or any carbon deposition on the surface of the catalyst. The results of FIGS. 10A-10F thereby demonstrate that the catalyst is resistant to coking and sintering.

Although particular embodiments have been shown and described, it will be appreciated by those skilled in the art that various changes and modifications might be made without departing from the scope of the disclosure. The terms and expressions used in the preceding specification have been used herein as terms of description and not of limitation, and there is no intention in the use of such terms and expressions of excluding equivalents of the features shown and described or portions thereof. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1. A catalyst for dry reforming of methane, comprising:
a catalyst composition comprising a nickel-copper mixture and a promoter, the promoter comprising barium.

2. The catalyst of claim 1, wherein nickel is present in the nickel-copper mixture at an atomic concentration of between about 20% and about 85%.

3. The catalyst of claim 1, wherein the barium is present in the catalyst composition at an atomic concentration of between about 0.1 and about 20%.

4. The catalyst of claim 1, wherein the barium is homogenously distributed in the nickel-copper mixture.

5. The catalyst of claim 1, wherein the nickel-copper mixture further comprises cobalt.

6. The catalyst of claim 1, wherein the catalyst composition further comprises at least one oxide additive.

7. The catalyst of claim 6, wherein the at least one oxide additive comprises zirconium oxide (ZrO₂), cerium oxide (CeO₂), praseodymium oxide (Pr6O11), a solid solution thereof, or a combination thereof.

8. The catalyst of claim 6, wherein the at least one oxide additive is present in the catalyst composition at a total volume percentage of about 5% to about 60%.

9. The catalyst of claim 6, wherein the at least one oxide additive is doped with aliovalent cations.

10. The catalyst of claim 1, wherein the promoter further comprises magnesium.

11. The catalyst of claim 1, further comprising a support for the catalyst composition.

12. The catalyst of claim 11, wherein the support comprises activated alumina or a soft fibrous insulation.

13. The catalyst of claim 11, wherein the catalyst composition is substantially homogenously distributed on the surface of the support.

14. A method for making a catalyst, comprising:
providing solutions of nickel, copper, and barium;
providing a support;
impregnating the support with the solutions to form an impregnated support;
heat treating the impregnated support to form a heat-treated support; and
activating the heat-treated support to form the catalyst.

15. The method of claim 14, wherein the step of impregnating the support with the solutions utilizes a vacuum or incipient wetness impregnation technique.

16. The method of claim 14, wherein activating the heat-treated support comprises reducing the heat-treated support in the presence of hydrogen.

17. The method of claim 14, further comprising providing at least one oxide additive solution, and wherein the step of impregnating the support with the solutions further comprises impregnating the support with the at least one oxide additive solution.

18. The method of claim 14, further comprising providing a cobalt solution and wherein the step of impregnating the support with the solutions further comprises impregnating the support with the cobalt solution.

19. A method for dry reforming of methane, comprising:
providing a catalyst comprising:
a catalyst composition comprising a nickel-copper mixture and a promoter, the promoter comprising barium; and
contacting a feed gas with the catalyst, the feed gas comprising methane and carbon dioxide.

20. The method of claim 19, wherein the step of contacting the feed gas with the catalyst is performed for at least 300 hours.
